(19) United States
(12) Patent Application Publication
(10) Pub. No.: US 2007/0197925 A1
(43) Pub. Date: Aug. 23, 2007

(54) Acquisition of multiple-lead electrocardiogram

(76) Inventor: Kenneth S. Moore, Pasadena, CA (US)

Correspondence Address: MCDERMOTT WILL & EMERY LLP, 18191 VON KARMAN AVE., SUITE 500, IRVINE, CA, 92612-7108, US

(21) Appl. No.: 11/359,308

(22) Filed: Feb. 17, 2006

(51) Int. Cl. A61B 5/04

(52) U.S. Cl. 600/509

(57) ABSTRACT

A system and method are disclosed to collect electrocardiograph data using a patient's hands. The patient's first hand is placed on a first and a second electrically conductive surface, such that the patient's first hand contacts portions of both the first and second electrically conductive surfaces and does not contact a third and a fourth electrically conductive surface. The patient's second hand is placed on the third and fourth electrically conductive surfaces, such that the patient's second hand contacts portions of both the third and fourth electrically conductive surfaces and does not contact the first and second electrically conductive surfaces. The electrically conductive surfaces are electrically isolated from one another and electrically connect the patient to an electrocardiograph. The ECG data is collected and displayed. The caregiver may use the resulting electrocardiogram to determine the patient's need for immediate medical treatment.

### BACKGROUND OF THE INVENTION

1. Field of the Invention

The present invention relates generally to electrocardiograms, and more particularly to a method and system of collecting electrocardiograph data.

2. Description of the Related Art

The electrocardiograph (ECG) is a well-known instrument used to record the electrical activity of the heart from the body surface of the patient. In general, the device is used to reveal disturbances in the electrical conduction pattern of the heart. The time course of each individual heartbeat gives rise to a repetitive waveform with characteristic P, Q, R, S, and T segments. These electrographic manifestations of the underlying heart activity have been attributed to the propagation of the electrical activity of the atria (P wave) through the conduction system to the depolarization waveform (QRS complex) of the ventricular tissues, followed by the repolarization of the ventricle, which gives rise to a characteristic waveform as well (T wave). The relationship between groups of beats permits rhythm analysis where tachyarrhythmias, bradyarrhythmias, and other abnormal rhythms can be readily discerned in the ECG waveform.

Typically, the electrocardiograph has 3-12 wire leads, which are attached to electrodes. The electrodes are placed at designated points on the patient's chest and limbs to conduct the electrical signals from the heart through the electrodes to the electrocardiograph for analysis and display. The display is known as an electrocardiogram and may be shown on an electronic display device, such as a cathode ray tube (CRT), or may be drawn by a strip recorder to produce a paper strip or rhythm strip, for example.

Prior to attaching the electrodes to the patient, the patient's clothing is removed from the patient's chest. To prepare the patient's skin for effective electrode adhesion, excess hair is removed from the electrode site. The skin is then cleaned and dried.

While this is conducive to obtaining a detailed electrocardiogram, the application of multiple electrodes to the skin is a time consuming procedure.

### SUMMARY OF THE INVENTION

There is a need for a device that acquires an electrocardiogram of a patient without the need to attach several leads and electrodes to the patient's body. There is a need for a device that allows an electrocardiogram to be performed quickly, such as in an emergency setting, or in a doctor's waiting room, for example, to permit caregivers to prioritize patients for medical treatment and to dispose harmful arrhythmias rapidly.

In an embodiment, the acquisition device comprises a plurality of electrically conductive surfaces and electrically connects a patient to an electrocardiograph. In another embodiment, the device comprises a plurality of electrically conductive surfaces and an electrocardiograph. In an embodiment, the device operates with three-lead electrocardiographs. In another embodiment, the device operates with electrocardiographs having greater than three leads.

In an embodiment, the acquisition device comprises support member and four electrically conductive surfaces on the upper surface of the support member. Each electrically conductive surface is substantially isolated from the other electrically conductive surfaces. The device further comprises four connectors, each connector capable of mating with a leadwire from an electrocardiograph. In an embodiment, the device further comprises signal-processing circuitry using well-known signal processing techniques. In a further embodiment, the acquisition device comprises a support member and three electrically conductive surfaces.

In an embodiment, a first electrically conductive surface electrically couples to a first connector, where the first connector electrically couples with a first leadwire input of the electrocardiograph. A second electrically conductive surface electrically couples to a second connector, where the second connector electrically couples with a second leadwire input of the electrocardiograph. A third electrically conductive surface electrically couples to a third connector, where the third connector electrically couples with a third leadwire input of the electrocardiograph. In an embodiment, the first leadwire input is a LA (left arm) leadwire input, the second leadwire input is a LL (left leg) leadwire input, and the third leadwire input is a RA (right arm) leadwire input of the electrocardiograph.

In an embodiment, a fourth electrically conductive surface is unconnected. In another embodiment, the fourth electrically conductive surface electrically connects to ground. In yet another embodiment, each electrocardiograph leadwire comprises a shield electrically connected to ground.

In an embodiment, the first, second, and third leadwire inputs of the electrocardiograph electrically couple to the first, second, and third conductive surfaces, respectively, through the signal-processing circuitry.

In an embodiment, a method of using the device comprises coupling the first connector to the LA leadwire input, coupling the second connector to the LL leadwire input, and coupling the third connector to the RA leadwire input of the electrocardiograph. The method further comprises coupling the device to the patient by placing the patient's first hand on the first and second electrically conductive surfaces. The patient's first hand contacts portions of both the first and second electrically conductive surfaces and does not contact the third and fourth electrically conductive surfaces. The method further comprises coupling the device to the patient by placing the patient's second hand on the third and fourth electrically conductive surfaces. The patient's second hand contacts portions of both the third and fourth electrically conductive surfaces and does not contact the first and second electrically conductive surfaces. The method further comprises printing or displaying the patient's electrocardiogram. The caregiver may use the resulting electrocardiogram to determine the patient's need for immediate medical treatment.

In an embodiment, an apparatus for electrically connecting a patient to an electrocardiograph comprises a first electrically conductive surface electrically coupled to a first connector, a second electrically conductive surface electrically coupled to a second connector, where the second conductive surface is substantially electrically isolated from the first conductive surface, and a third electrically conductive surface electrically coupled to a third connector, where the third conductive surface is substantially electrically isolated from the first and second surfaces. The apparatus further comprises a support member, where the first and second conductive surfaces are supported by the support member. The first connector is configured to couple to a first leadwire input of an electrocardiograph, the second connector is configured to couple to a second leadwire input of the electrocardiograph, and the third connector is configured to couple to a third leadwire input of the electrocardiograph. The first and second conductive surfaces are disposed so as to permit simultaneous contact by a first human extremity, and the third conductive surface is disposed so as to permit contact by a second human extremity.

In an embodiment, a method of collecting electrocardiograph data comprises contacting with a patient's first hand a first electrically conducting surface and a second electrically conducting surface. The first electrically conducting surface electrically couples to a first leadwire input of an electrocardiograph, the second conducting surface electrically couples to a second leadwire input of the electrocardiograph, and the first electrically conducting surface is substantially electrically isolated from the second electrically conducting surface. The method further comprises contacting with the patient's second hand a third electrically conducting surface, where the third electrically conducting surface electrically couples to a third leadwire input of the electrocardiograph, and where the third electrically conducting surface is substantially electrically isolated from the first and second electrically conducting surfaces.

In an embodiment, an apparatus for electrically connecting a patient to an electrocardiograph comprises a first means for sensing electrical activity of a patient's first hand, and a second means for sensing electrical activity of the patient's first hand, where the first means for sensing electrically couples to a first leadwire input of an electrocardiograph and the second means for sensing electrically couples to a second leadwire input of the electrocardiograph, and where the first means for sensing is substantially electrically isolated from the second means for sensing. The apparatus further comprises a means for supporting the first and second means for sensing. The apparatus further comprises a third means for sensing electrical activity of a patient's second hand, where the third means for sensing electrically couples to a third leadwire input of the electrocardiograph, and where the third means for sensing is substantially electrically isolated from the first and second means for sensing.

For purposes of summarizing the invention, certain aspects, advantages, and novel features of the invention have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1 is a perspective view of an embodiment of the acquisition device.

FIG. 2 is a perspective view of another embodiment of the acquisition device.

FIG. 3 is a system block diagram of an embodiment of a system for the acquisition of a three-lead electrocardiogram.

FIG. 4 is a system block diagram of another embodiment of the system for the acquisition of a three-lead electrocardiogram.

FIG. 5 illustrates the collection of ECG data, according to an embodiment of the invention.

FIG. 6 illustrates a portion of an exemplary electrocardiogram.

FIG. 7 is a flow chart illustrating the collection of ECG data, according to an embodiment of the invention.

### DETAILED DESCRIPTION OF THE PREFERRED EMBODIMENT

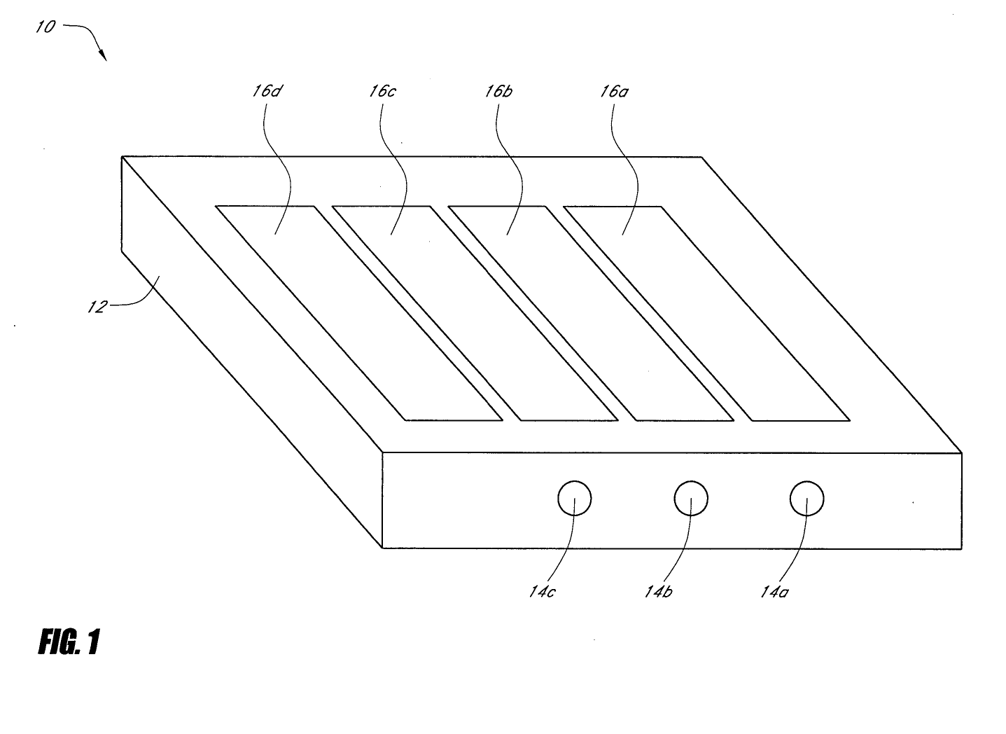

For a more detailed understanding of the invention, reference is first made to FIG. 1. FIG. 1 is a perspective view of an embodiment of a three-lead electrocardiogram acquisition device 10 comprising a support structure or member 12, connectors 14, and conductive contact pads 16. The support structure 12 comprises a non-conductive material and supports the contact pads 16, such that the contact pads 16 are electrically isolated from each other. In an embodiment, the support structure 12 comprises a support platform or platform member, such as a piece of wood, non-conductive plastic, other non-conductive material, or the like. In other embodiments, the support structure 12 comprises struts, stilts, standoffs, or the like.

In an embodiment, the support structure 12 comprises a non-conductive box-like structure. In other embodiments, the support structure can be round, oval, square, rectangular, or other substantially two-dimensional shapes, cylindrical, cubic, spherical, other substantially three-dimensional shapes, or the like. Examples of electrically non-conductive materials are plastic, wood, ABS, polystyrene, fiberglass, polyester, PVC, blended plastic, polycarbonate, styrene, and the like. The support structure 12 is sized to accommodate comfortably a patient's hand or hands placed flat, palms down, and side-by-side on the upper surface of the support structure 12.

The electrically conductive contact pads 16 are mounted on the upper surface of the support structure 12. As is known to one of skill in the art, the conductive pads or plates 16 can be associated with the support member 12 in other ways, such as etched into the support member 12, deposited into or onto the support member 12, integrated into the support member, or the like.

Each electrically conductive contact pad 16 is substantially electrically isolated from each of the other electrically conductive pads 16. Examples of electrically conductive pad materials are metal, conductive plastics, and the like. The conductive contact pads 16 are sized such that the patient's first hand, placed on the upper surface of the enclosure, contacts the conductive contact pads 16a and 16b and does not contact the conductive contact pads 16c and 16d, while the patient's second hand, placed on the upper surface of the enclosure, contacts the conductive contact pads 16c and 16d, and does not contact the conductive contact pads 16a and 16b. In an embodiment, the acquisition device 10 comprises three conductive contact pads 16. In another embodiment, the acquisition device 10 comprises more than three conductive contact pads 16.

In a further embodiment, the acquisition device 10 comprises two conductive pads 16 for contacting the patient's hand, where the two conductive pads are supported by the support member 12.

Connectors 14 comprise, for example, snap connectors, pinch connectors, standard electrocardiograph lead connectors, and the like, which are configured to mate with standard electrocardiograph leadwires. In an embodiment, the acquisition device 10 comprises three connectors 14. In another embodiment, the acquisition device 10 comprises more than three connectors 14.

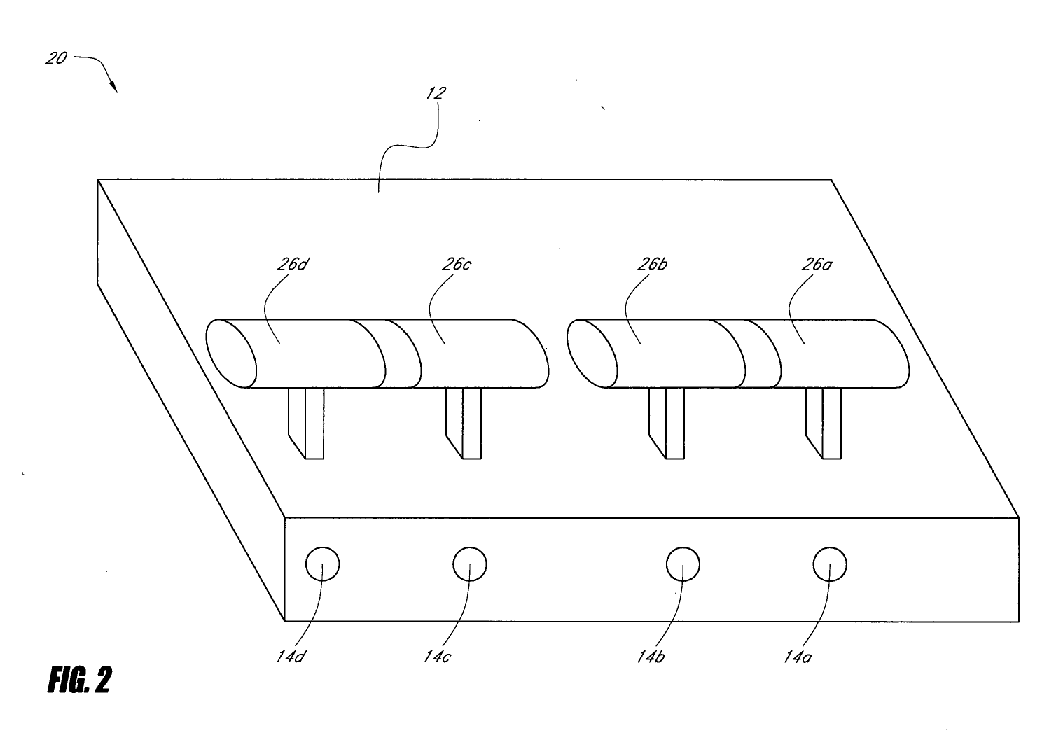

FIG. 2 is a perspective view of another embodiment of a two-lead acquisition device 20 comprising the support structure or member 12, the connectors 14, and conductive contact grips 26. Each electrically conductive contact grip 26 is substantially electrically isolated from each of the other electrically conductive grips 26. Examples of electrically conductive grip materials are metal, conductive plastics, and the like. The conductive contact grips 26 are sized such that the patient's first hand can grip a handle on the upper surface of the support structure 12 formed of conductive contact grips 26a and 26b and not contact conductive contact grips 26c and 26d, while the patient's second hand can grip a handle on the upper surface of the support structure 12 formed of the conductive contact grips 26c and 26d and not contact the conductive contact grips 26a and 26b.

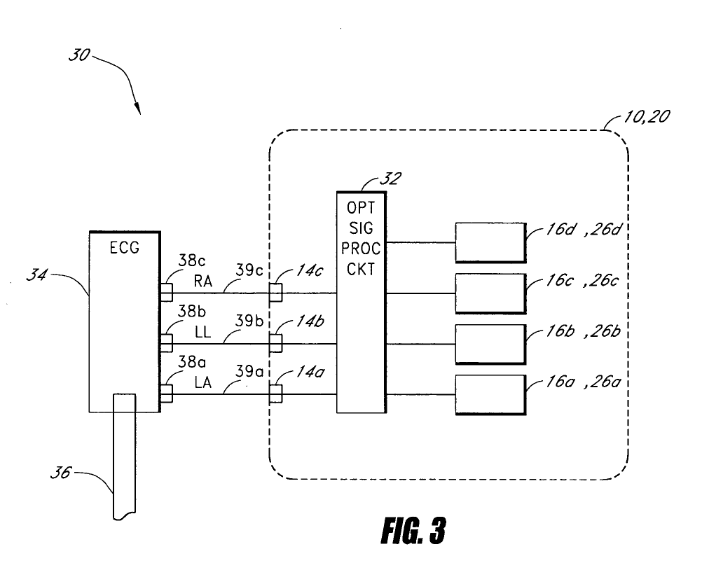

FIG. 3 is a system block diagram of an embodiment of a system 30 for the acquisition of a three-lead electrocardiogram comprising the acquisition device 10, 20, and an electrocardiograph 34. In an embodiment, the acquisition device 10, 20 comprises the conductive contact pads/grips 16, 26, the connectors 14, and an optional signal processing block 32.

The optional signal processing block 32 comprises signal processing circuitry as known to one of skill in the art. In an embodiment, the signal processing block 32 comprises electronic signal processing circuitry for reducing signal distortion due to electronic noise. In another embodiment, the signal processing circuitry is configured to improve the signal to noise ratio of an electrical signal conducted through the signal processing circuitry. In another embodiment, the signal processing block 32 comprises a computer, such as a digital signal processor, and computer instructions, which are executed by the digital signal processor to reduce distortion caused by electronic noise.

The electrocardiograph 34 comprises a plurality of leadwire inputs 38. Leadwires 39 connect to the leadwire inputs 38 and the connectors 14 to couple the electrocardiograph 34 to the acquisition device 10, 20. The electrocardiograph 34 produces a visual representation of the patient's electrical heart activity. In an embodiment, the electrocardiograph 34 comprises a monitor, such as an LCD, an LED, or a CRT display. In another embodiment, the electrocardiograph 34 comprises a strip recorder and produces a paper chart or rhythm strip 36 of the patient's electrical heart activity.

As illustrated in FIG. 3, the conductive contact/grip 16a, 26a electrically couples via the optional signal processing block 32 through the connector 14a to a first leadwire input 38a of the electrocardiograph 34. In an embodiment, the first leadwire input 38a comprises the left arm (LA) leadwire input of the electrocardiograph 34.

Similarly, the conductive contact/grip 16b, 26b electrically couples via the optional signal processing block 32 through the connector 14b to a second leadwire input 38b of the electrocardiograph 34. In an embodiment, the second leadwire input 38b comprises the left leg (LL) leadwire input of the electrocardiograph 34.

Likewise, conductive contact pad/grip 16c, 26c electrically couples via the optional signal processing block 32 through the connector 14c to a third leadwire input 38c of the electrocardiograph 34. In an embodiment, the third leadwire input 38c comprises the right arm (RA) leadwire input of the electrocardiograph 34.

In an embodiment, the conductive contact pad/grip 16d, 26d electrically connects to the optional signal processing block 32. In another embodiment, the conductive contact pad/grip 16d, 26d is electrically open, such that the conductive contact pad/grip 16d, 26d is not electrically connected to the electrocardiograph 34 or the optional signal processing block 32. In yet another embodiment, the conductive contact pad/grip 16d, 26d connects to ground.

In yet a further embodiment, the conductive contact pad/grip 16d, 26d electrically couples via the optional signal processing block 32 through the connector 14d to a fourth leadwire input (not shown) of the electrocardiograph 34. In an embodiment, the fourth leadwire input comprises the right leg (RL) leadwire input of the electrocardiograph 34.

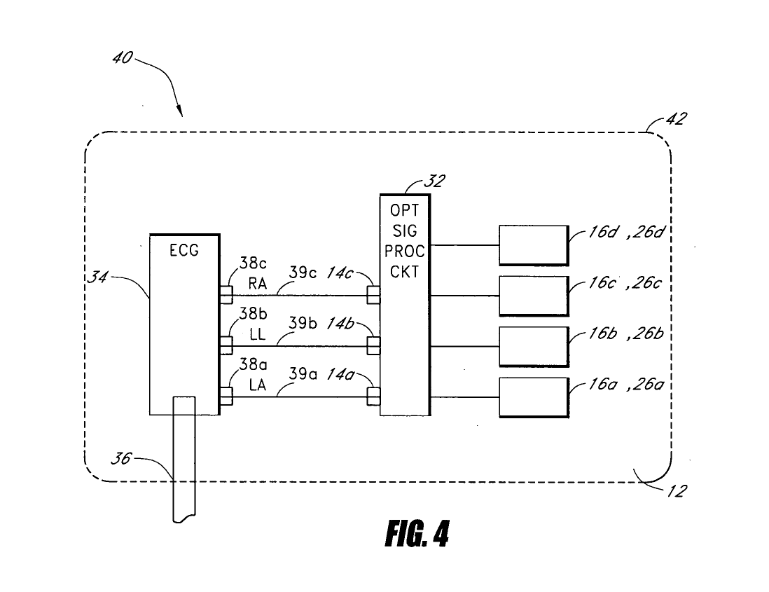

FIG. 4 is a system block diagram of another embodiment of a system 40 for the acquisition of a three-lead electrocardiogram. The system 40 comprises the acquisition device 42. The acquisition device 42 comprises the connectors 14, the conductive contact pads/grips 16, 26, the optional signal processing block 32, and the electrocardiograph 34. The electrocardiograph 34 comprises the electrocardiograph leadwire inputs 38 and a display, such as the rhythm strip 36. The connectors 14, the conductive contact pads/grips 16, 26, the optional signal processing block 32, the electrocardiograph 34, and the electrocardiograph leadwire inputs 38 are connected as described above with respect to the acquisition system 30 of FIG. 3.

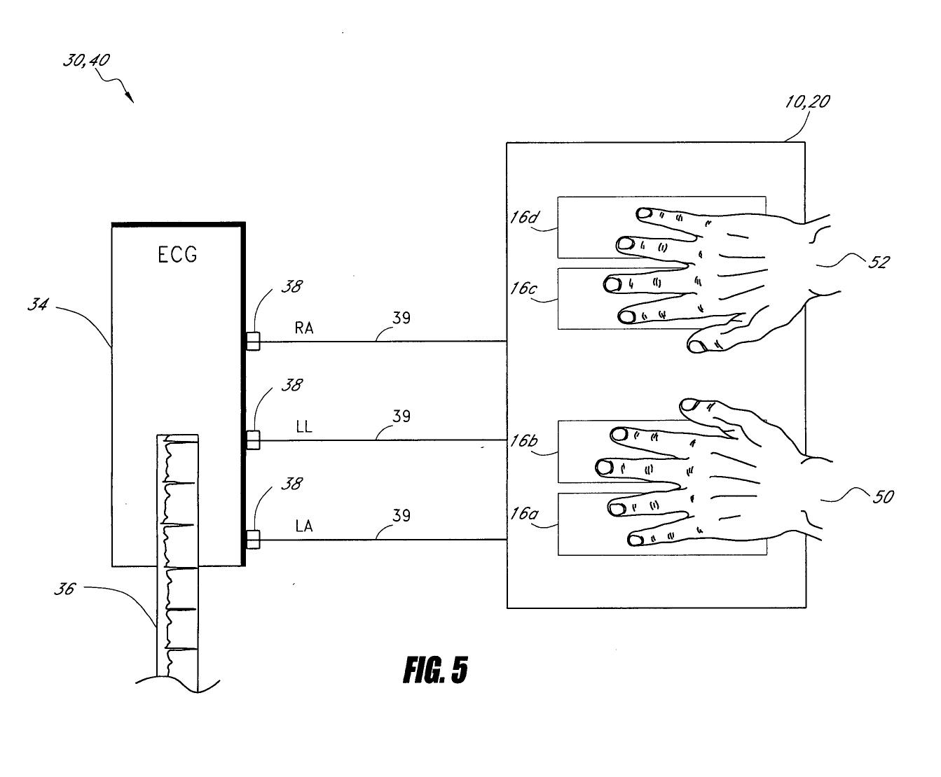

FIG. 5 illustrates the collection of ECG data using the acquisition system 30, 40, according to an embodiment of the invention. The patient's first hand 50 contacts the conductive contact pads 16a and 16b and does not contact the conductive contact pads 16c and 16d. The patient's second hand 52 contacts the conductive contact pads 16c and 16d and does not contact the conductive contact pads 16a and 16b.

The conductive contact pads 16 detect the signals from the patient's heart activity. The electrocardiograph 34 measures the potential difference between the signals on the leadwire inputs 38 caused by the electrical excitation generated by the patient's cardiac muscle. The electrocardiograph 34 produces the record 36 of the patient's heart activity from the patient's body surface.

In an embodiment, where the patient's hands 50, 52 are placed on the conductive contact pads 16a, 16b, 16c or the patient grips the conductive grips 26a, 26b, 26c, and the contact pads/grips 16a/26a, 16b/26b, 16c/26c are electrically coupled to the LA, LL, and RA leadwire inputs, respectively, of the electrocardiograph 34, as described above, the contact pads/grips 16, 26 comprise three measurement points. The three measurement points produce three leads, which can be defined as:

Lead A: H₁=φ_{LA}-φ_{RA}

Lead B: H₂=φ_{LL}-φ_{RA}

Lead C: H₃=φ_{LL}-φ_{LA}

where: H₁=the voltage of Lead A
H₂=the voltage of Lead B
H₃=the voltage of Lead C
φ_{LA}=potential at a first point on the patient's left hand
φ_{LL}=potential at a second point on the patient's the left hand
φ_{RA}=potential at a first point on the patient's right hand

The electrocardiograph 34 measures the electrical potentials of the patient, converts the electrical potential differences to a readable electrocardiogram reading by calculating the leads A, B, and C, and records the repetitive waveform 36 indicative of the electrical activity of the patient's heart. In an embodiment, the electrocardiograph 34 displays one of leads A, B, or C. The operator of the electrocardiograph 34 selects the lead A, B, or C that provides the most readable electrocardiogram for display.

In a conventional application of an electrocardiograph, leads I, II, and III are calculated from the electrical signals on the LA, LL, and RA, leadwire inputs, which in a conventional application are connected to electrodes located on the patient's left arm, left leg, and right arm, respectively. Leads A, B, and C do not provide the same information as in the conventional application of the electrocardiograph. Leads A, B, and C do not provide the same spatial information as leads I, II, and III in a normal electrocardiogram, as the electrodes are not connected to the same anatomical structure.

In an embodiment, selecting lead A for the display 36 corresponds to the operator selecting lead I on the electrocardiograph 34 and the electrocardiograph senses the signals from the RA and LA leadwires. Selecting lead B for the display 36 corresponds to the operator selecting lead II on the electrocardiograph 34 and the electrocardiograph senses the signals from the RA and LL leadwires. Selecting lead C for the display 36 corresponds to the operator selecting lead III on the electrocardiograph 34 and the electrocardiograph senses the signals from the LA and LL leadwires.

The labels LA, LL, RA, and RL are used herein to indicate that the electrical signals acquired by the data acquisition device 10, 20 are input into the electrocardiograph 34 through the LA, LL, RA, and RL leadwire inputs 38.

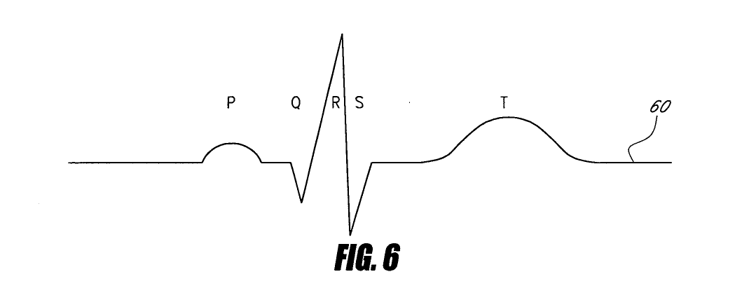

FIG. 6 illustrates a portion of an exemplary electrocardiogram 60 comprising the P, QRS, and T complexes of the waveform. The electrocardiogram 60 is useful in diagnosing abnormal heart rhythms, such as, premature ventricular contractions, dysrhythmia, atrial fibrillation, ventricular fibrillation, heart blockage, atrial flutter, bradycardia, tachycardia, atrial tachycardia, ventricular tachycardia, and the like.

The acquisition device 10, 20 provides a quick way to obtain the patient's heart rhythm to permit prioritization of medical treatment according to the seriousness of the patient's condition or injury as indicated by the patient's electrocardiogram 36. In an embodiment, the acquisition device 10, 20 is used in a doctor's office to prioritize medical treatment. In another embodiment, the acquisition device is used in an emergency setting, such as a hospital emergency department, or the site of an accident, natural disaster, or battlefield, to ration limited medical resources when the number of injured needing care exceeds the resources available to perform care so as to treat the greatest number of patients possible, i.e. triage.

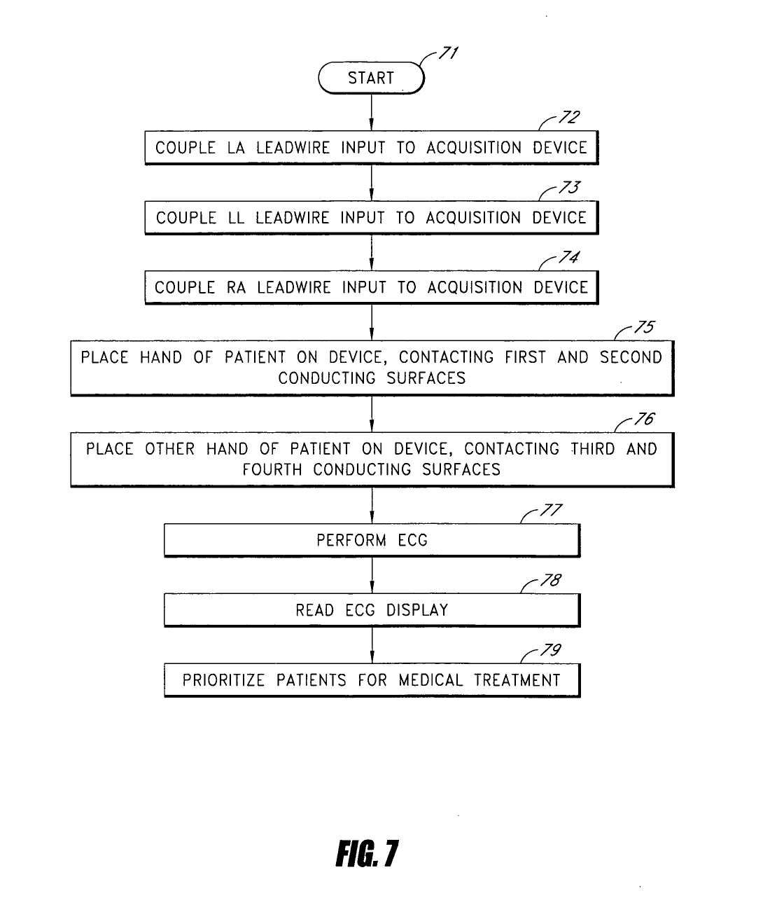

FIG. 7 is a flow chart 70 illustrating the collection of ECG data, according to an embodiment of the invention. From a start block 71, the left arm (LA) leadwire input 38a from the electrocardiograph 34 couples to the connector 14a on the device 10, 20 in block 72. In block 73, the left leg (LL) leadwire input 38b from the electrocardiograph 34 couples to the connector 14b on the device 10, 20. In block 74, the right arm (RA) leadwire input 38c from the electrocardiograph 34 couples to the connector 14c on the device 10, 20.

In block 75, the patient's first hand 50 is placed on the conductive contact pads 16a and 16b. In block 76, the patient's second hand 52 is placed on conductive contact pads 16c and 16d. In another embodiment, other contact areas of the patient's body can be used, such as, for example, the patient's feet. In another embodiment, the patient's first hand 50 is placed on a first acquisition device 10 comprising the two contact pads 16a and 16b and the patient's second hand 52 is placed on a second acquisition device 10 comprising the two contact pads 16c and 16d.

In block 77, the electrocardiograph 34 acquires the electrical signals from the activation of the patient's cardiac muscle through the contact of the patient's body surface on the conductive contact pads/grips 16, 26. The electrocardiograph 34 produces the electrocardiogram 36.

In block 78, the electrocardiogram 36 is read and analyzed by the medical personnel.

In block 79, the patient is prioritized for medical treatment based, at least in part on the electrocardiogram 36.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions, and changes in the form of the methods and systems described herein may be made without departing from the spirit of the inventions. The accompanying claims and their equivalents are intended to cover such forms or modifications as would fall within the scope and spirit of the inventions.

What is claimed is:

1. An apparatus for electrically connecting a patient to an electrocardiograph comprising:
a first electrically conductive surface electrically coupled to a first connector;
a second electrically conductive surface electrically coupled to a second connector, wherein the second conductive surface is substantially electrically isolated from the first conductive surface;
a third electrically conductive surface electrically coupled to a third connector, wherein the third conductive surface is substantially electrically isolated from the first and second surfaces; and
a support member, wherein the first and second conductive surfaces are supported by the support member;
wherein the first connector is configured to couple to a first leadwire input of an electrocardiograph, wherein the second connector is configured to couple to a second leadwire input of the electrocardiograph, and wherein the third connector is configured to couple to a third leadwire input of the electrocardiograph; and
wherein the first and second conductive surfaces are disposed so as to permit simultaneous contact by a first human extremity, and the third conductive surface is disposed so as to permit contact by a second human extremity.

2. The apparatus of claim 1 further comprising a fourth electrically conductive surface electrically coupled to a fourth connector;
wherein the fourth conductive surface is substantially electrically isolated from the first, second, and third conductive surfaces;
wherein the fourth connector is configured to connect to a fourth leadwire input of the electrocardiograph; and
wherein the third and fourth conductive surfaces are disposed so as to permit simultaneous contact by the second human extremity.

3. The apparatus of claim 2, wherein the fourth leadwire input comprises a right leg (RL) leadwire input.

4. The apparatus of claim 1, wherein the third conductive surface is supported by the support member.

5. The apparatus of claim 1, wherein the conductive surfaces are attached to the support member.

6. The apparatus of claim 1, wherein the first leadwire input comprises a left arm (LA) leadwire input, the second leadwire input comprises a left leg (LL) leadwire input, and the third leadwire input comprises a right arm (RA) leadwire input.

7. The apparatus of claim 1 further comprising the electrocardiograph.

8. The apparatus of claim 1 further comprising signal processing circuitry in electrical communication with the first, second, and third conductive surfaces, wherein the signal processing circuitry is configured to improve a signal to noise ratio of an electrical signal conducted through the signal processing circuitry.

9. The apparatus of claim 8, wherein the signal processing circuitry comprises a first signal processing circuit, a second signal processing circuit, and a third signal processing circuit.

10. The apparatus of claim 9, wherein an input of the first signal processing circuit electrically couples to the first conducting surface and an output of the first signal processing circuit electrically couples to the first connector.

11. The apparatus of claim 10, wherein an input of the second signal processing circuit electrically couples to the second conducting surface and an output of the second signal processing circuit electrically couples to the second connector.

12. The apparatus of claim 11, wherein an input of the third signal processing circuit electrically couples to the third conducting surface and an output of the third signal processing circuit electrically couples to the third connector.

13. A method of collecting electrocardiograph data comprising:
contacting with a patient's first hand a first electrically conducting surface and a second electrically conducting surface, wherein the first electrically conducting surface electrically couples to a first leadwire input of an electrocardiograph and the second conducting surface electrically couples to a second leadwire input of the electrocardiograph, and wherein the first electrically conducting surface is substantially electrically isolated from the second electrically conducting surface; and
contacting with the patient's second hand a third electrically conducting surface, wherein the third electrically conducting surface electrically couples to a third leadwire input of the electrocardiograph, and wherein the third electrically conducting surface is substantially electrically isolated from the first and second electrically conducting surfaces.

14. The method of claim 13 further comprising collecting and displaying electrocardiogram data from the patient.

15. The method of claim 14 further comprising signal processing the electrocardiogram data.

16. The method of claim 13 further comprising contacting with the patient's second hand the third electrically conducting surface and a fourth electrically conducting surface, wherein the fourth electrically conducting surface is substantially electrically isolated from the first, second, and third electrically conducting surfaces.

17. An apparatus for electrically connecting a patient to an electrocardiograph comprising:
first means for sensing electrical activity of a patient's first hand;
second means for sensing electrical activity of the patient's first hand;
wherein the first means for sensing electrically couples to a first leadwire input of an electrocardiograph and the second means for sensing electrically couples to a second leadwire input of the electrocardiograph, and wherein the first means for sensing is substantially electrically isolated from the second means for sensing;
means for supporting said first and second means for sensing; and
third means for sensing electrical activity of a patient's second hand;
wherein the third means for sensing electrically couples to a third leadwire input of the electrocardiograph, and wherein the third means for sensing is substantially electrically isolated from the first and second means for sensing.

18. The apparatus of claim 17 further comprising a fourth means for sensing electrical activity of the patient's second hand, wherein the fourth means for sensing is substantially electrically isolated from the first, second, and third means for sensing.

19. The apparatus of claim 18, wherein the fourth means for sensing electrically couples to a fourth leadwire input of the electrocardiograph.

20. The apparatus of claim 17 further comprising a support means, wherein the first, second, and third means for sensing are located on a surface of the support means.

21. The apparatus of claim 17 further comprising the electrocardiograph.
